(19) United States
(12) Patent Application Publication
(10) Pub. No.: US 2009/0215756 A1
(43) Pub. Date: Aug. 27, 2009

(54) FORMULATIONS CONTAINING LOSARTAN AND/OR ITS SALTS

(75) Inventor: Sergio Lloret Perez, Barcelona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: COMBINO PHARM, S.L., Barcelona, ES (ES)

(21) Appl. No.: 11/914,828

(22) PCT Filed: May 17, 2006
(86) PCT No.: PCT/IB2006/003440

§ 371 Date: Jul. 14, 2008

Related U.S. Application Data
(60) Provisional application No. 60/681,961, filed on May 18, 2005.

(51) Int. Cl. A61K 31/5415; C07D 403/08; A61P 9/12

(52) U.S. Cl. 514/224.2; 548/252

(57) ABSTRACT

The invention relates, in general, to new formulations and dosage units containing losartan and/or its salts (e.g., losartan potassium) that are useful for the therapeutic treatment (including prophylactic treatment) of mammals, including humans, and a process for making the same. The process generally includes (i) mixing and blending losartan potassium and a first portion of at least one of pre-gelatinized starch, microcrystalline cellulose and magnesium stearate to the mixture of step (i); (ii) compacting the mixture obtained in step (i) to form an agglomerate; (iii) breaking apart the agglomerate in order to obtain a granulate; (iv) adding lactose monohydrate and a second portion of at least one of pre-gelatinized starch, microcrystalline cellulose and magnesium stearate to the granulate and blending; and (v) tableting the granulate mixture into tablets. The process can further include coating the prepared tablets with a suitable coating material.

### CROSS REFERENCE TO RELATED APPLICATIONS

This application claims priority to U.S. Provisional Application No. 60/681,961, filed May 18, 2005, which application is expressly incorporated herein by reference in its entirety.

### BACKGROUND OF THE INVENTION

1. Field of the Invention

The invention relates, in general, to new formulations and dosage units containing losartan and/or its salts (e.g., losartan potassium) that are useful for the therapeutic treatment (including prophylactic treatment) of mammals, including humans, and a process for making the same.

2. Relevant Background

Losartan free acid is also known as 2-butyl-4-chloro-1-[[(2′-(1H-tetrazol-5-yl)[1,1′-biphenyl]-4-yl]methyl]-1H-imidazole-5-methanol, whose potassium salt has been shown to be useful in the treatment of hypertension.

Losartan potassium has been approved by the FDA for the treatment of hypertension. The product is marketed as coated tablets for oral administration under the name Cozaar®, and in combination with hydrochlorothiazide as coated tablets for oral administration, under the trade mark of Hyzaar®.

Losartan may be prepared using the reactions and techniques described in U.S. Pat. No. 5,138,069, U.S. Pat. No. 5,130,439 and U.S. Pat No. 5,206,374, the disclosures of which are incorporated herein by reference.

It has been observed that there can be difficulties preparing tablets containing losartan potassium by direct compression when the losartan potassium has an approximately 90% distribution (by volume) of particle sizes below approximately 30 μm. Namely, when losartan potassium having these particle sizes is used, there can, for example, be flowability problems with the compression mixtures, and/or the mixtures can exhibit sticking and picking phenomena with the dye and upper punch respectively. Such difficulties can result in weight and hardness variations in the obtained tablets.

Another aspect of the invention is to provide a process for preparing formulations and dosage units containing losartan and/or its salts (e.g., losartan potassium) that includes a pre-compression or compacting step in which losartan potassium is compressed with some of the excipients to be included in the final formulation and where the losartan potassium has a particle size distribution in which approximately 90% (by volume) of the particles have a diameter below 30 μm. The process of the invention is, however, applicable to losartan and/or its salts (e.g., losartan potassium) having a particle size distribution in which approximately 90% of the particles have a diameter above approximately 45 μm and/or 50 μm.

### SUMMARY OF THE INVENTION

The invention relates, in general, to new formulations and dosage units containing losartan and/or its salts (e.g., losartan potassium) that are useful for the therapeutic treatment (including prophylactic treatment) of mammals, including humans, and a process for making the same. The process generally includes (i) mixing and blending losartan potassium and a first portion of at least one of pre-gelatinized starch, microcrystalline cellulose and magnesium stearate to the mixture of step (i); (ii) compacting the mixture obtained in step (i) to form an agglomerate; (iii) breaking apart the agglomerate in order to obtain a granulate; (iv) adding lactose monohydrate and a second portion of at least one of pre-gelatinized starch, microcrystalline cellulose and magnesium stearate to the granulate and blending; and (v) tableting the granulate mixture into tablets. The process can further include coating the prepared tablets with a suitable coating material.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the dissolution profile of a 100 mg formulation of losartan potassium from Example 2 and the in vitro drug release/dissolution profile of a marketed formulation (100 mg tablet) of losartan potassium (i.e., Cozaar®); and

FIG. 2 illustrates the in vitro drug release/dissolution profile for the losartan potassium tablet (100 mg) obtained in Example 4 compared to that of a marketed formulation (100 mg tablet) of losartan potassium (i.e., Cozaar®).

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention includes a process for formulating losartan and/or its salts (e.g. losartan potassium) into readily usable dosage units for the therapeutic treatment (including prophylactic treatment) of mammals, including humans. Such formulations are normally formulated in accordance with standard pharmaceutical practice as a pharmaceutical composition.

The formulations prepared by the process of the invention can include alternative equivalent excipients (i.e., other release control agents, fillers, lubricants and/or binders) having the same and/or similar functions and/or properties may be readily substituted and used in the above illustrative formulation. Additional suitable pharmaceutically-acceptable excipients for a tablet formulation include, for example, crospovidone, silicon dioxide, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid.

The two-step process of the invention can advantageously be adapted to utilize losartan and/or its salts (e.g., losartan potassium) having a particle size distribution in which approximately 90% of the particles have a diameter below approximately 30 μm, which includes conducting a pre-compression or compacting step in which losartan potassium is compacted with some of the excipients to be included in the final formulation. The process of the invention is, however, applicable to losartan and/or its salts (e.g., losartan potassium) having a particle size distribution in which approximately 90% of the particles have a diameter above approximately 45 μm and/or 50 μm.

During the development of this process, it was observed that the composition of the product of the pre-compression or compacting step was determinative for dissolution speed and for avoiding flowability and compression problems. Thus, changes in the composition of the product of the pre-compression or compacting step enables the dissolution profile to be modulated and flowability and compression problems to be avoided. It has been further observed that inclusion and/or exclusion of lactose in the pre-compression or compacting step is critical to the characteristics (i.e., dissolution profile) of the product of the pre-compression or compacting step.

According to another aspect of the invention, the process for preparing formulations and dosage units containing losartan and/or its salts (e.g., losartan potassium) generally includes: (i) weighing, sieving, mixing and blending losartan potassium, a first portion of pre-gelatinized starch and a first portion of microcrystalline cellulose; (ii) adding a first portion of magnesium stearate to the mixture of step (i) and blending the mixture to ensure good homogeneity in a suitable blending apparatus (e.g., a tumbling blender, V-blender or bin blender); (iii) compacting and milling the mixture to form a granulation (e.g., by adding the blended mixture to the hopper of a roller compacter); (iv) adding and blending any additional excipients as required/desired and sieving the resulting mixture (e.g., adding lactose monohydrate, an additional portion of pre-gelatinized starch, an additional portion of microcrystalline cellulose and an additional portion of magnesium stearate to the granulate and blending); and (v) forming tablets using the product obtained in steps (iv).

The obtained tablet formulations and dosage units may be uncoated or coated, either to modify their disintegration and the subsequent absorption of the active ingredient or to improve their stability and/or appearance, using conventional coating agents and procedures well known in the art. For example, a standard water-based coating process can be performed in a suitable coating pan or fluid bed.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

### Specific Examples

The following examples are for illustrative purposes only and are not intended, nor should they be interpreted, to limit the scope of the invention.

General Experimental Conditions: Dissolution Profiles

Dissolution profiles were obtained according to the following analytical method. The obtained tablets (100 mg) of Examples 2 and4 and commercially available losartan potassium tablets (100 mg) were tested for in vitro drug release in 900 mL of USP dissolution medium pH 6.8. A USP-2 apparatus with paddle speed at 50 rpm was used for the study. The amount of dissolved losatan was determined conventionally by HPLC using a suitable chromatographic column (e.g., a Kromasil C18 5 μm 25 cm×4.6 mm column) with a mobile phase consisting of 1100 mL of potassium dihydrogen phosphate buffer, pH 3.0 and 900 mL of acetonitrile, and a flow rate of approximately 1.2 mL/min. at room temperature. Detection was accomplished using UV absorption at 254 nm. Data is quantified by comparison of the HPLC peak area relative to the peak area taken from a standard plot of concentration versus peak area for standards of known concentration In this regard, losartan standard concentrations are selected to fall within a range of concentration versus absorbance for the UV detector employed.

### EXAMPLE 1

### Formulations of Losartan Potassium Tablets (100 mg)

Table 1 illustrates a preferred tablet formulation containing losartan and/or its salts (e.g., losartan potassium) according to the process of the invention.

TABLE 1

| Per Tablet (mg) | Ingredient |
|---|---|
| 100.00 | Losartan Potassium |
| 51 | Lactose Monohydrate |
| 41.90 | Starch (pregelatinized) |
| 105 | Microcrystalline Cellulose |
| 2.10 | Magnesium Stearate |
| 300.00 | TOTAL (CORE) |
| | Coating Mixture |
| 3.60 | Hydroxypropylmethylcellulose |
| 3.60 | Hydroxypropylcellulose |
| 111.00 | Deionized Water |
| 1.80 | Titanium Dioxide |
| 0.05 | Micronized Carnauba Wax |
| 309.05 | TOTAL (TABLET) |

### EXAMPLE 2

### Process for Preparing Formulation of Losartan Potassium Tablets (100 mg)

As discussed above, the invention includes a two-step process (i.e., one involving both a pre-compression or compacting step and a compression step) for preparing cores containing losartan and/or its salts (e.g., losartan potassium), such as those described in Table 1. In particular, the formulation described in Table 1 can be prepared according to this two-step process as described below in Table 2.

TABLE 2

| | | |
|---|---|---|
| Per Tablet (mg) | Ingredient | Procedure Comment |

STEP 1

| | | |
|---|---|---|
| 100.00 | Losartan Potassium | Total amount used in compacting step |
| 80.00 | Microcrystalline Cellulose (1) | ~76% of total amount used in tablet |
| 31.50 | Pre-gelatinized Starch (1) | ~75% of total amount used in tablet |
| 0.20 | Magnesium Stearate (1) | ~10% of total amount used in tablet |

STEP 2

| | | |
|---|---|---|
| 51.00 | Lactose Monohydrate | Total amount used in tablet |
| 25.00 | Microcrystalline Cellulose (2) | ~24% of total amount used in tablet |
| 10.40 | Pre-gelatinized Starch (2) | ~25% of total amount used in tablet |
| 1.90 | Magnesium Stearate (2) | ~90% of total amount used in tablet |
| 300.00 | TOTAL CORE | |
| 3.60 | Hydroxypropylmethylcellulose | Coating component |
| 3.60 | Hydroxypropylcellulose | Coating component |
| 95.00 | Purified Water¹ | Coating component |
| 1.80 | Titanium Dioxide | Coating component |
| 16.00 | Purified Water¹ | Coating component |
| 0.05 | Carnauba Wax (micronized) | |
| 309.05 | TOTAL COATED TABLET | |

Table 2 Note:
¹The purified water disappears during the manufacturing process

The cores of the formulation of Table 2 were prepared in the following steps: (i) mixing and blending the losartan potassium, a first portion of the pre-gelatinized starch and a first portion of the microcrystalline cellulose; (ii) adding a first portion of the magnesium stearate to the blended mixture and further blending the mixture; (iii) compacting the blended mixture to obtain an agglomerate; (iv) breaking the agglomerate in order to obtain a granulate; (v) adding lactose monohydrate and the remaining pre-gelatinized starch and the remaining microcrystalline cellulose to the granulate and blending the mixture; (vi) adding the remaining magnesium stearate to the blended mixture; (vii) compressing the blended mixture into tablets; and (viii) coating the tablets with the above coating material until their a weight increased by approximately 3%.

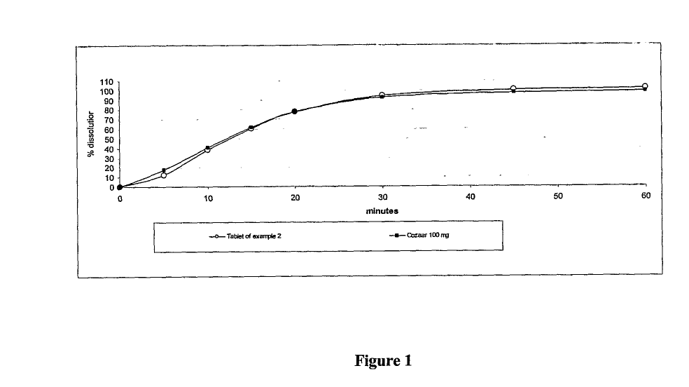

FIG. 1 illustrates the dissolution profile of a 100 mg formulation of losartan potassium from Example 2 and the in vitro drug release/dissolution profile of a marketed formulation (100 mg tablet) of losartan potassium (i.e., Cozaar®), and shows that these formulations have a similar dissolution profile.

Notably, during pre-compression or compacting step no flowability and compression problems were observed.

The formulations of Tables 1 and 2 is proportional and scaleable for a tablet containing 50 mg of losartan potassium.

### EXAMPLE 3

### Formulation of Losartan Potassium Tablets (100 mg)

Table 3 illustrates a tablet formulation containing losartan and/or its salts (e.g., losartan potassium) in which lactose was included in the pre-compression or compacting step.

TABLE 3

| | |
|---|---|
| Per Tablet (mg) | Ingredient |

STEP 1

| | |
|---|---|
| 100.00 | Losartan Potassium |
| 40.0 | Lactose Monohydrate (1) |
| 31.5 | Pre-gelatinized Starch (1) |
| 80.0 | Microcrystalline Cellulose (1) |
| 0.20 | Magnesium Stearate (1) |

STEP 2

| | |
|---|---|
| 11.0 | Lactose Monohydrate (2) |
| 10.4 | Pre-gelatinized Starch (2) |
| 25.0 | Microcrystalline Cellulose (2) |
| 1.90 | Magnesium Stearate (2) |
| 300.00 | TOTAL CORE |

The formulation of Table 3 was prepared for tablet manufacture by the following process: (i) mixing and blending the losartan potassium, a first portion of the lactose monohydrate, a first portion of the pre-gelatinized starch and a first portion of the microcrystalline cellulose; (ii) adding a first portion of the magnesium stearate to the blended mixture and further blending the mixture; (iii) compacting the blended mixture to obtain an agglomerate; (iv) breaking the agglomerate in order to obtain a granulate; (v) adding the remaining lactose monohydrate, the remaining pre-gelatinized starch and the remaining microcrystalline cellulose to the granulate and blending; (vi) adding the remaining magnesium stearate to the granulate mixture and blending the mixture; and (vii) compressing the blended mixture into tablets.

Notably, flowability and compression problems were observed during the pre-compression or compacting step.

### EXAMPLE 4

### Formulation of Losartan Potassium Tablet Cores (100 mg)

Table 4 illustrates another tablet formulation containing losartan and/or its salts (e.g., losartan potassium) in which lactose was included in the pre-compression or compacting step.

TABLE 4

| | |
|---|---|
| Per Tablet (mg) | Ingredient |

STEP 1

| | |
|---|---|
| 100.00 | Losartan Potassium |
| 25.50 | Lactose Monohydrate (1) |
| 20.95 | Pre-gelatinized Starch (1) |
| 52.50 | Microcrystalline Cellulose (1) |
| 0.20 | Magnesium Stearate (1) |

STEP 2

| | |
|---|---|
| 25.50 | Lactose Monohydrate (2) |
| 20.95 | Pre-gelatinized Starch (2) |
| 52.50 | Microcrystalline Cellulose (2) |
| 1.90 | Magnesium Stearate (2) |
| 300.00 | TOTAL CORE |
| 3.60 | Hydroxypropylmethylcellulose |
| 3.60 | Hydroxypropylcellulose |
| 95.00¹ | Purified water (a) |
| 1.80 | Titanium Dioxide |
| 16.00¹ | Purified Water (b) |
| 0.05 | Carnauba Wax (micronized) |
| 309.05 | TOTAL COATED TABLET |

Table 4 Note:
¹The purified water disappears during the manufacturing process

The formulation of Table 4 was prepared for tablet manufacture by the following process: (i) mixing and blending the losartan potassium, a first portion of the lactose monohydrate, a first portion of the pre-gelatinized starch and a first portion of the microcrystalline cellulose; (ii) adding a first portion of the magnesium stearate to the blended mixture and further blending the mixture; (iii) compacting the blended mixture to obtain an agglomerate; (iv) breaking the agglomerate in order to obtain a granulate; (v) adding the remaining lactose monohydrate, the remaining pre-gelatinized starch and the remaining microcrystalline cellulose (to the granulate and blending; (vi) adding the remaining magnesium stearate to the granulate mixture and blending the mixture; and (vii) compressing the blended mixture into tablets.

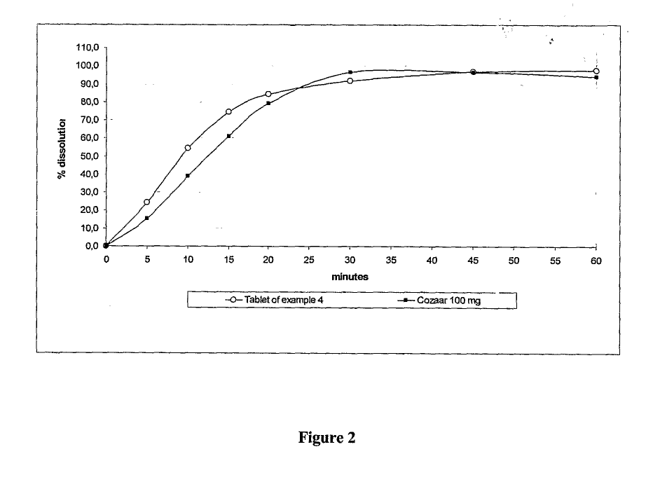

Notably, flowability and compression problems were observed during the pre-compression or compacting step. FIG. 2 illustrates the in vitro drug release/dissolution profile for the losartan potassium tablet (100 mg) obtained in Example 4 compared to that of a marketed formulation (100 mg tablet) of losartan potassium (i.e., Cozaar®) and shows that these formulations have a different dissolution profile.

### EXAMPLE 5

### Formulation of Losartan Potassium Tablet Cores (100 mg)

Table 5 illustrates another tablet formulation containing losartan and/or its salts (e.g., losartan potassium) in which lactose was included in the pre-compression or compacting step.

TABLE 5

| | |
|---|---|
| Per Tablet (mg) | Ingredient |

STEP 1

| | |
|---|---|
| 100.00 | Losartan Potassium |
| 11.0 | Lactose Monohydrate (1) |
| 19.5 | Pre-gelatinized Starch (1) |
| 20.0 | Microcrystalline Cellulose (1) |
| 0.20 | Magnesium Stearate (1) |

STEP 2

| | |
|---|---|
| 40.0 | Lactose Monohydrate (2) |
| 22.4 | Pre-gelatinized Starch (2) |
| 80.0 | Microcrystalline Cellulose (2) |
| 1.90 | Magnesium Stearate (2) |
| 300.00 | TOTAL CORE |

The formulation of Table 5 was prepared for tablet manufacture by the following process: (i) mixing and blending the losartan potassium, a first portion of the lactose monohydrate, a first portion of the pre-gelatinized starch and a first portion of the microcrystalline cellulose; (ii) adding a first portion of the magnesium stearate to the blended mixture and further blending the mixture; (iii) compacting the blended mixture to obtain an agglomerate; (iv) breaking the agglomerate in order to obtain a granulate; (v) adding the remaining lactose monohydrate, the remaining pre-gelatinized starch and the remaining microcrystalline cellulose (to the granulate and blending; (vi) adding the remaining magnesium stearate to the granulate mixture and blending the mixture; and (vii) compressing the blended mixture into tablets.

Notably, flowability and compression problems were observed during the pre-compression or compacting step.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. A process for preparing a formulation of a pharmaceutically acceptable quantity of losartan and/or its pharmaceutically acceptable salts comprising:
(i) mixing and blending losartan potassium and a first portion of at least one of pre-gelatinized starch, microcrystalline cellulose and magnesium stearate;
(ii) compacting the mixture obtained in step (i) to form an agglomerate;
(iii) breaking apart said agglomerate obtained in step (ii) in order to obtain a granulate;
(iv) adding lactose monohydrate and a second portion of at least one of pre-gelatinized starch, microcrystalline cellulose and magnesium stearate to said granulate and blending; and
(v) tableting the granulate mixture obtained in step (iv) into tablets.

2. The process of claim 1, further comprising coating said tablets with a coating material.

3. The process of claim 1, wherein said coating material comprises at least one of hydroxypropylmethylcellulose, hydroxypropylcellulose, deionized water, titanium dioxide and micronized carnauba wax.

4. The process of claim 2, further comprising coating said tablets with a coating material until the weight of said tablets increases by approximately 3%.

5. The process of claim 1, further comprising the addition of at least one additional excipient material.

6. The process of claim 5, wherein said at least one additional excipient material is at least one of a lactose monohydrate/corn starch mixture, lactose, lactose (anhydrous), sodium carbonate, calcium phosphate, calcium carbonate, corn starch, algenic acid, starch, stearic acid, talc, crospovidone, silicon dioxide ethyl p-hydroxybenzoate, propyl p-hydroxybenzoate and ascorbic acid.

7. The process of claim 1, wherein said losartan potassium has a particle size distribution in which approximately 90% of the particles have a diameter between approximately 30 μm and approximately 50 μm.

8. The process of claim 1, wherein said losartan potassium has a particle size distribution in which approximately 90% of the particles have a diameter less than approximately 30 μm.

9. The process of claim 1, wherein in step (i) said first portion of microcrystalline cellulose comprises approximately 76% by weight of the total amount of microcrystalline cellulose included in said tablet, said first portion of pre-gelatinized starch comprises approximately 75% by weight of the total amount of pre-gelatinized starch included in said tablet and said first portion of magnesium stearate comprises approximately 10% by weight of the total amount of magnesium stearate included in said tablet.

10. The process of claim 1, wherein in step (iv) said lactose monohydrate comprises approximately 100% by weight of the total amount of lactose monohydrate included in said table said second portion of pre-gelatinized starch comprises approximately 25% by weight of the total amount of pre-gelatinized starch included in said tablet, said second portion of microcrystalline cellulose comprises approximately 24% by weight of the total amount of microcrystalline cellulose included in said tablet and said second portion of magnesium stearate comprises approximately 90% by weight of the total amount of magnesium stearate included in said tablet.

11. A formulation of a pharmaceutically acceptable quantity of losartan and/or its pharmaceutically acceptable salts comprising a tablet, wherein said tablet is prepared according to the process of claim 1.

12. The formulation of claim 11, further comprising a coating.

13. The formulation of claim 11, wherein said tablet comprises approximately 100.00 mg of losartan potassium, approximately 51.00 mg of lactose monohydrate, approximately 41.90 mg of starch, approximately 105.00 mg of microcrystalline cellulose and approximately 2.10 mg of magnesium stearate.
